(19) United States
(12) Patent Application Publication
(10) Pub. No.: US 2025/0383417 A1
(43) Pub. Date: Dec. 18, 2025

(54) Homogenization of Magnetic Resonance Data

(71) Applicant: Siemens Healthineers AG, Forchheim (DE)

(72) Inventors: Wei Liu, Erlangen (DE); Muhammed Labeeb Kozhikkavil, Erlangen (DE); Nicolas Groß-Weege, Nürnberg (DE)

(73) Assignee: Siemens Healthineers AG, Forchheim (DE)

(21) Appl. No.: 19/235,761

(22) Filed: Jun. 12, 2025

(30) Foreign Application Priority Data
Jun. 14, 2024 (DE) 10 2024 205 490.9

(51) Int. Cl. G01R 33/56; A61B 5/055; G16H 50/30

(52) U.S. Cl. CPC G01R 33/5608; A61B 5/055; G16H 50/30

(57) ABSTRACT

Method for generating homogenized magnetic resonance data, including: providing magnetic resonance data of an examination object; providing a first homogenization field specific to the examination object; providing a frequency spectrum having frequency ranges; spectral decomposition of the magnetic resonance data into the frequency ranges, wherein first magnetic resonance data is associated with a first frequency range of the frequency ranges and second magnetic resonance data is associated with a second frequency range of the frequency ranges; determining a second homogenization field by taking into account the first homogenization field; applying the first homogenization field to the first magnetic resonance data and generating first homogenized magnetic resonance data; applying the second homogenization field to the second magnetic resonance data and generating second homogenized magnetic resonance data; and generating homogenized magnetic resonance data by combining the first homogenized magnetic resonance data with the second homogenized magnetic resonance data.

### TECHNICAL FIELD

The disclosure relates to a method, an image processing device, a computer program product, as well as an electronically readable data carrier for generating homogenized magnetic resonance data.

### BACKGROUND

In a magnetic resonance device, the body to be examined of an examination object, in particular of a patient, is customarily exposed with the aid of a main magnet to a main magnetic field, for example, of 1.5 or 3, or 7 tesla. In addition, gradient pulses are played out with the aid of a gradient coil unit. High-frequency radio frequency pulses, for example excitation pulses, are then emitted via a radio frequency antenna unit by means of suitable antenna facilities, and this results in the nuclear spins of specific atoms resonantly excited by these radio frequency pulses being tilted by a defined flip angle with respect to the magnetic field lines of the main magnetic field. On relaxation of the nuclear spins, radio frequency signals, which are known as magnetic resonance signals, are irradiated which are received by means of suitable radio frequency antennas and then processed further. From the raw data thus acquired, it is finally possible to reconstruct the desired image data. The region of the examination object to be represented is the examination region. The examination object is typically a human, in particular a patient.

For a specific measurement, a specific magnetic resonance control sequence (MR control sequence), also called a pulse sequence, should therefore be emitted which is composed of a series of radio frequency pulses, for example excitation pulses and refocusing pulses, as well as gradient pulses which are to be emitted so as to be appropriately coordinated therewith in different gradient axes along different spatial directions. Readout windows that are appropriate time-wise are set, which specify the periods in which the induced MR signals are captured.

A magnetic field gradient, in particular a temporary magnetic field gradient, is generated in the examination region when a gradient pulse is emitted. A temporary RF field, also called a B1 field, B1+field, or B1 sending field, is generated in the examination region when an excitation pulse is emitted. Similarly, the radio frequency antennas have a spatial characteristic, which is referred to as a B1 receiving field or B1 field, suitable for receiving the MR signals irradiated by the examination object. The B1 field and the B1 receiving field can be influenced in various ways in the examination region by the geometry of the examination object and/or owing to a design of a component of the magnetic resonance device, and be inhomogeneous in various ways. The same applies to the main magnetic field and/or magnetic field gradient. This can result in a tissue having different signal intensities in image data. Therefore, the image data is corrupted. A homogenization field, known in the specialist literature as a “bias field”, is a spatially resolved measured representative of gentle changes in the signal intensity owing to these effects. Changes in the signal intensity owing to these effects and possible further influences typically correspond to a local reduction in the signal intensity, so image data can be darker locally and/or can have local effacements. A homogenization field can quantify locally varying signal intensities in image data. Using the homogenization field, raw data or the reconstructed image data can be homogenized. A homogenization of magnetic resonance data can comprise a correction of a local variation in a signal intensity, which local variation in a signal intensity is not based on an anatomy and/or pathology of the examination object and/or the examination region. For each image point and/or for each voxel within the examination region, the homogenization field typically comprises a correction value, corresponding to a scalar, which comprises a relative amplification and/or attenuation for a measured signal intensity in image data. A conventional homogenization of image data comprises a multiplication or division of the signal intensity of an image data point by the corresponding correction value of the homogenization field. The homogenization field is typically individual to each examination object and each examination region. In the context of the disclosure, it has been found that a conventional homogenization can adversely affect the noise in image data.

### SUMMARY

The disclosure is based on a method for a particularly comprehensive homogenization of magnetic resonance data. The object is achieved by the features of the independent claims. Advantageous aspects are described in the subclaims.

The inventive method for generating homogenized magnetic resonance data provides the following method steps:
providing magnetic resonance data of an examination object,
providing a first homogenization field specific to the examination object,
providing a frequency spectrum having at least two frequency ranges,
spectral decomposition of the magnetic resonance data into the at least two frequency ranges, wherein first magnetic resonance data is associated with a first frequency range of the at least two frequency ranges and second magnetic resonance data is associated with a second frequency range of the at least two frequency ranges,
determining a second homogenization field by taking into account the first homogenization field,
applying the first homogenization field to the first magnetic resonance data and generating first homogenized magnetic resonance data,
applying the second homogenization field to the second magnetic resonance data and generating second homogenized magnetic resonance data,
generating homogenized magnetic resonance data by combining the first homogenized magnetic resonance data with the second homogenized magnetic resonance data.

The magnetic resonance data can comprise raw data. The magnetic resonance data can comprise image data and/or raw data reconstructed to form image data. Providing the magnetic resonance data can comprise capturing the magnetic resonance data, in particular, an acquisition of magnetic resonance data by means of a magnetic resonance device. Providing the magnetic resonance data can comprise a transfer of the magnetic resonance data from a memory unit to an image processing unit.

Providing the first homogenization field can comprise capturing the first homogenization field, in particular, an acquisition by means of a magnetic resonance device. Providing the first homogenization field can comprise a transfer of the first homogenization field from a memory unit to an image processing unit.

The examination object is typically a patient from which image data is created within the framework of a magnetic resonance examination for a clinical diagnosis. The magnetic resonance data and/or the first homogenization field can be provided, in particular recorded, within the framework of the magnetic resonance examination. The magnetic resonance data and the first homogenization field are typically specific to the examination object, in particular to the examination region of the examination object. The first homogenization field can be embodied specifically for the magnetic resonance data.

Providing the frequency spectrum having at least two frequency ranges can comprise a division of the frequency spectrum into at least two frequency ranges. Providing the frequency spectrum can comprise a transfer of the frequency spectrum from a memory unit to an image processing unit. The frequency spectrum typically comprises a frequency range including a spectrum of conventional magnetic resonance data. The at least two frequency ranges are preferably disjunct.

Image data is typically present in the image data space. The image data space typically comprises image points and/or voxels, with magnetic resonance data embodied as image data comprising a signal intensity for each image point of the examination region. Raw data is typically present in the raw data space, also called k-space. Owing to the acquisition methods of the raw data by utilizing different frequency modulations, it is collected in the raw data space. The raw data space can be transformed into the image data space by means of Fourier transform and/or inverse Fourier transform. The raw data can be transformed into image data by means of Fourier transform and/or inverse Fourier transform.

The spectral decomposition of the magnetic resonance data into the at least two frequency ranges can comprise filtering of the magnetic resonance data in such a way that, firstly, by means of filters, the low-frequency, for example, the first magnetic resonance data associated with the first frequency range, is extracted from the magnetic resonance data. Low-frequency first magnetic resonance data of this kind reproduces gross changes in the signal intensity, such as, for example, owing to anatomical structures. The second magnetic resonance data, which is to be associated with the higher second frequency range, can be determined by subtraction of the first magnetic resonance data from the magnetic resonance data. High-frequency second magnetic resonance data of this kind can reproduce abrupt changes in the signal intensity owing to noise, therefore also at edges of anatomical structures. This procedure is advantageous, in particular, if the magnetic resonance data is embodied as image data.

If the magnetic resonance data is embodied as raw data, the magnetic resonance data can thus be spectrally decomposed by the division of the raw data space into at least two frequency ranges. Thus, for example, the first frequency range can comprise the periphery of the raw data space, and the second frequency range the center of the raw data space. The second frequency range can also comprise the periphery of the raw data space, and the first frequency range the center of the raw data space. The center of the raw data space can comprise, for example, the central 30% of the raw data, i.e., the 30% of the raw data that is closest to the center of the raw data space. The periphery of the raw data space can comprise the raw data space minus the center.

If the magnetic resonance data is embodied as image data, the spectral decomposition can thus comprise a transformation, for example Fourier transform, of the magnetic resonance data into the raw data space, whereby the transformed magnetic resonance data is embodied as raw data. The spectral decomposition can take place analogously to the magnetic resonance data embodied as raw data. The union of the first magnetic resonance data with the second magnetic resonance data typically forms the magnetic resonance data.

Applying the first homogenization field typically comprises a voxel-wise multiplication and/or division of the first magnetic resonance data by the first homogenization field. If the first magnetic resonance data is embodied as raw data, applying the first homogenization field to the first magnetic resonance data thus typically also comprises a transformation in advance of the first magnetic resonance data into the image space, so the first magnetic resonance data can be multiplied and/or divided voxel-wise by the first homogenization field in the image space.

Determining the second homogenization field typically comprises a modification of the first homogenization field. Applying the second homogenization field typically comprises a voxel-wise multiplication and/or division of the second magnetic resonance data by the second homogenization field. If the second magnetic resonance data is embodied as raw data, applying the second homogenization field to the second magnetic resonance data thus typically also comprises a transformation in advance of the second magnetic resonance data into the image space, so the second magnetic resonance data can be multiplied and/or divided voxel-wise by the second homogenization field in the image space.

The generation of homogenized magnetic resonance data by combining the first homogenized magnetic resonance data with the second homogenized magnetic resonance data can take place, for example, by means of voxel-wise addition. The first homogenized magnetic resonance data, the second homogenized magnetic resonance data, and the homogenized magnetic resonance data are typically embodied as image data.

The inventive method makes homogenization of the magnetic resonance data possible as a function of the spectrum of the magnetic resonance data. Magnetic resonance data can thus be associated with corresponding frequency ranges and homogenized in different ways. Noise in magnetic resonance data typically occurs in a dedicated frequency range which depends, for example, on the MR control sequence used and/or the voxel size. By using a second homogenization field, the inventive method makes it possible for corresponding second magnetic resonance data in a dedicated second frequency range to experience a different homogenization than the first magnetic resonance data outside of the second frequency range. This makes a comprehensive homogenization of the magnetic resonance data possible. In addition, the inventive method is independent of the homogenization field used and/or the contrast of the magnetic resonance data, and can be flexibly implemented.

One aspect of the method provides that the first frequency range has lower frequencies than the second frequency range. The spectral decomposition of the first magnetic resonance data accordingly has lower frequencies than the spectral decomposition of the second magnetic resonance data. According to this aspect, the first magnetic resonance data has smooth changes in the signal intensity, free from sharp edges and/or free from abrupt changes in the contrast. According to this aspect, first magnetic resonance data of this kind is homogenized using the first homogenization field. Preferably, the first homogenization field corresponds to a conventional homogenization field used within the framework of homogenization, so the low-frequency first magnetic resonance data is conventionally homogenized.

According to this aspect, the second magnetic resonance data has a higher-frequency component, i.e., for example, abrupt changes in the signal intensity owing to noise and/or due to edges of anatomical structures. The second magnetic resonance data typically comprises the noise, i.e., the component of the signal intensity of the magnetic resonance data that is caused by noise. According to this aspect, second magnetic resonance data of this kind is homogenized by using the second homogenization field. The second homogenization field is preferably embodied in such a way that signal intensities in the second magnetic resonance data caused by noise are amplified less compared to the first magnetic resonance data.

It has been found that the noise in the examination region is typically uniformly distributed whereas the homogenization field typically varies as a function of sensitivities in the receive coils with distance from the surface of the body in the direction of the inside of the body and/or has a gradient over at least parts of the examination region. It has been found that portions of the examination region, which are subject within the framework of a conventional homogenization of an above average amplification of the measured signal intensity, after homogenization owing to the above average amplification of the noise, have a lower signal-to-noise ratio than portions with below average amplification of the measured signal intensity. It has been found that portions of the examination region, which are spaced apart further from the surface of the body of the examination object, typically have a lower signal-to-noise ratio than portions arranged closer to the surface of the body of the examination object. According to this aspect, the inhomogeneity of the signal-to-noise ratios, in particular, can be improved over the examination region. This aspect prevents an amplification of the noise in the homogenized magnetic resonance data.

One aspect of the method provides that the second homogenization field is determined by taking into account the second magnetic resonance data. The second homogenization field typically comprises a modification of the first homogenization field, with the second magnetic resonance data being taken into account in the case of modification according to this aspect. In particular, signal intensities incorporated by the second magnetic resonance data and/or a spatial distribution of the second magnetic resonance data in the examination region can be taken into account. This makes a particularly individual generation of second homogenized magnetic resonance data possible, so noise present in the second magnetic resonance data can be reduced particularly effectively.

One aspect of the method provides that determining the second homogenization field comprises the following method steps:
providing a mapping rule mapping first correction values of the first homogenization field to second correction values,
generating the second homogenization field by applying the mapping rule to the first homogenization field.

For each image point in the examination region, the first homogenization field typically comprises a first correction value. The homogenization of the first magnetic resonance data, i.e., generating the first homogenized magnetic resonance data, typically comprises a voxel-wise multiplication of the first magnetic resonance data by the corresponding first correction value. A first value range typically comprises all first correction values that the first homogenization field provides for the image points in the examination region. For each image point in the examination region, the second homogenization field typically comprises a second correction value. The homogenization of the second magnetic resonance data, i.e., generating the second homogenized magnetic resonance data, typically comprises a voxel-wise multiplication of the second magnetic resonance data by the corresponding second correction value.

The mapping rule typically associates a second correction value with each first correction value. The mapping rule is typically independent of individual image points. The mapping rule is typically a scalar function that associates a second correction value with a first correction value, with the first correction value and the second correction value each being scalar. The mapping rule typically has a positive derivation.

Applying the mapping rule to the first homogenization field provides that the mapping rule is applied pixel-wise, i.e., separately for each image point: the mapping rule is applied to the first correction value of an individual image point and generates the second correction value, thereby for this image point. The second homogenization field can be generated by corresponding repetition for all image points of the examination region.

The use of a mapping rule makes homogenization possible not only by taking into account the frequencies of the magnetic resonance data, which makes separation of the noise together with other radio-frequency signals possible, but also taking into account the intensity of the sought correction. For example, if the second frequency range comprises lower frequencies than the first frequency range, according to this aspect, the amplification can be deliberately reduced or increased for low-frequency magnetic resonance signals with a high signal intensity for which the first homogenization field provides a low amplification. If the second frequency range has higher frequencies than the first frequency range, the second magnetic resonance data thus typically comprises noise and sharp edges of anatomical structures, which typically have different signal intensities. Sharp edges typically have a higher signal intensity than the noise and thereby a lower first correction value.

The mapping rule can be selected in such a way that high first correction values are attenuated compared to lower first correction values.

According to this aspect, magnetic resonance data can be homogenized particularly diversely by taking into account individual noise. In particular, the weighting of the homogenization with regard to frequency and signal intensity can be effectively implemented.

One aspect of the method provides that the mapping rule is provided by taking into account the second magnetic resonance data. In this case, the signal intensities provided for image points by the second magnetic resonance data are typically taken into account before their homogenization, hereinafter referred to as second signal intensities. In particular, the value range of the second signal intensities and/or the second signal intensities can be taken into account. Preferably, the signal intensities provided for image points by the first magnetic resonance data are also taken into account before their homogenization, hereinafter referred to as the first signal intensities, which intensities correlate with the first correction values. In particular, the value range of the second signal intensities can be compared with the value range of the first signal intensities. In addition and/or alternatively, the second signal intensities can also be compared with the first signal intensities. Alternatively, the fraction of the first homogenization field relevant to the second magnetic resonance data can also be taken into account. If the second magnetic resonance data comprises, for example, above-average or below-average signal intensities, the mapping rule can thus explicitly expand or compress them. This makes a particularly good homogenization of the magnetic resonance data possible.

One aspect of the method provides that a second value range comprising the second correction values is smaller than a first value range comprising the first correction values. According to this aspect, the spectrum of the correction of the second magnetic resonance data is accordingly smaller than the spectrum of the correction of the first magnetic resonance data. This is advantageous, in particular, if the first frequency range has lower frequencies than the second frequency range, since high-frequency noise is modulated less as a result and the noise of the homogenized magnetic resonance data is thereby more homogeneous.

One aspect of the method provides that a first value range comprising the first correction values comprises a first section and a second section, the first section comprises greater first correction values than the second section and the gradient of the mapping rule in the first section is smaller than the gradient of the mapping rule in the second section.

The first section and the second section are typically disjunct portions of the first value range. The first section and the second section are typically a connected quantity respectively. According to this aspect, the first section comprises higher first correction values than the second section. According to this aspect, the mean of the first correction values of the first section is higher than the mean of the first correction values of the second section. The first section accordingly comprises the first correction values, which are provided for first magnetic resonance data with lower first signal intensity, as the lower first correction values of the second section, which are provided for first magnetic resonance data with higher first signal intensity. The first section and the second section can be separated from each other by a first threshold value. The first threshold value can preferably be manually and/or individually determined within the framework of providing the mapping rule.

According to this aspect, the mapping rule is embodied in such a way that the gradient of the mapping rule in the first section is smaller than the gradient of the mapping rule in the second section. The gradient, in particular the mathematical derivation, of the mapping rule in the first section and in the second section is typically positive. Preferably, the mean of the gradient of the mapping rule in the first section is smaller than the mean of the gradient of the mapping rule in the second section. The gradient of the mapping rule in the second section can be embodied to be constant or can vary within the second section. The gradient of the mapping rule in the first section can be embodied to be constant or can vary within the first section. The gradient of the mapping rule can be continuous at the transition from the first section to the second section and/or have a constant derivation.

The gradient of the mapping rule in the first section and/or the mean of the gradient of the mapping rule in the first section is typically between 0.01 and 0.8, preferably between 0.05 and 0.5, particularly preferably between 0.1 and 0.3. The gradient of the mapping rule in the second section and/or the mean of the gradient of the mapping rule in the second section is typically between 0.8 and 1.2, preferably between 0.9 and 1.1, particularly preferably between 0.95 and 1.05.

This aspect accordingly provides that high first correction values are reduced for low first signal intensities in the context of the mapping rule, so low second signal intensities are amplified less by application of the second homogenization field compared to low first signal intensities. If the second frequency range comprises higher frequencies than the first frequency range and/or the second magnetic resonance data is higher frequency than the first magnetic resonance data, the second magnetic resonance data thus typically comprises the noise that a low first signal intensity has. According to this aspect, it is amplified by a reduced second correction value, and this reduces the noise in the second homogenized magnetic resonance data and thereby in the homogenized magnetic resonance data.

One aspect of the method provides that the first section comprises at least one portion of the upper third of the first value range, and/or the second section comprises at least one portion of the middle third of the first value range. The first section can also comprise the upper quarter of the first value range or a portion of the upper quarter of the first value range. An aspect of this kind of the mapping rule makes particularly good homogenization of magnetic resonance data possible, for which a high amplification within the framework of homogenization is provided.

One aspect of the method provides that a first value range comprising the first correction values comprises a third section and a fourth section, the third section comprises greater first correction values than the fourth section and the mapping rule is embodied in such a way that the second correction values of the first section associated with the first correction values are greater than the corresponding first correction values of the first section.

The third section and the fourth section are typically disjunct portions of the first value range. The third section and the fourth section are typically a connected quantity respectively. According to this aspect, the third section comprises higher first correction values than the fourth section. According to this aspect, the mean of the first correction values of the third section is higher than the mean of the first correction values of the fourth section. The third section accordingly comprises the first correction values, which for first magnetic resonance data are provided with a lower first signal intensity than the lower first correction values of the fourth section, which for first magnetic resonance data are provided with a higher first signal intensity. The third section and the fourth section can be separated from each other by a second threshold value. The second threshold value can preferably be manually and/or individually determined within the framework of providing the mapping rule.

According to this aspect, the mapping rule is embodied in such a way that, preferably, a second correction value is allocated to each first correction value of the fourth section by the mapping rule, which second correction value is greater than the first correction value. The gradient, in particular the mathematical derivation, of the mapping rule in the third section and in the fourth section is typically positive.

This aspect accordingly provides that low first correction values for high first signal intensities are increased in the context of the mapping rule, so high second signal intensities are amplified more compared to high first signal intensities by the application of the second homogenization field. If the second frequency range comprises higher frequencies than the first frequency range and/or the second magnetic resonance data are higher frequency than the first magnetic resonance data, the second magnetic resonance data thus typically comprises edges owing to anatomical structures, such as, for example, sharp transitions between different textures, which have a high contrast in the magnetic resonance data. Thus, for example, the transition from muscle tissue, having low signal intensity, to a fat layer adjoining the muscle tissue, having high signal intensity, can represent a high local difference in the signal intensity, which corresponds to a high-frequency signal and can be associated with the second magnetic resonance data within the framework of the spectral decomposition.

According to this aspect, image points located at edges in underlying structures and having high signal intensities, such as, for example, fat adjoining muscle tissue, can be amplified with an increased second correction value, and this makes a distinction of such anatomical signal variations from noise in the second homogenized magnetic resonance data possible and thereby spatially homogenizes the signal-to-noise ratio in the homogenized magnetic resonance data.

One aspect of the method implements the above aspect in that the mapping rule in the fourth section provides, for example, a gradient that is smaller than the gradient of the mapping rule in the third section. The gradient of the mapping rule in the fourth section can be embodied to be constant or can vary within the fourth section. The gradient of the mapping rule in the third section can be embodied to be constant or can vary within the third section. The gradient of the mapping rule can be continuous at the transition from the third section to the fourth section and/or have a constant derivation. In addition, the mapping rule at the transition from the third section to the fourth section can be embodied to be steady. The gradient of the mapping rule in the third section and/or the mean of the gradient of the mapping rule in the third section is typically between 0.8 and 1.2, preferably between 0.9 and 1.1, particularly preferably between 0.95 and 1.05.

One aspect of the method provides that the gradient of the mapping rule in the fourth section is greater than the gradient of the mapping rule in the third section. According to this aspect, the mapping rule is preferably embodied in such a way that the gradient of the mapping rule in the fourth section and/or the mean of the gradient of the mapping rule in the fourth section is greater than 1. The gradient of the mapping rule in the fourth section and/or the mean of the gradient of the mapping rule in the fourth section can be between 1 and 5, typically between 1.1 and 3, preferably between 1.5 and 2.5, particularly preferably 2. The gradient of the mapping rule in the fourth section can be embodied to be constant or can vary within the fourth section. The gradient of the mapping rule in the third section can be embodied to be constant or can vary within the third section. The mapping rule can be embodied to be unsteady at the transition from the third section to the fourth section. This aspect makes particularly high amplification of low second signal intensities and thereby a particularly good distinction of high anatomical signal variations from noise in the second homogenized magnetic resonance data possible.

One aspect of the method provides that the fourth section comprises at least one portion of the lower third of the first value range, and/or the third section comprises at least one portion of the middle third of the first value range. The fourth section can also comprise the lower quarter of the first value range or a portion of the lower quarter of the first value range. An aspect of this kind of the mapping rule makes particularly good homogenization of magnetic resonance data possible, for which low amplification within the framework of homogenization is provided.

One aspect of the method provides that the third section corresponds to the second section. According to this aspect, the mapping rule has a different gradient at the edges of the first value range than in the middle third. In particular, especially high second signal intensities of the second magnetic resonance data are consequently amplified more or attenuated less than if they had been associated with the first magnetic resonance data during the spectral decomposition. In addition, particularly low second signal intensities of the second magnetic resonance data are amplified less or attenuated more than if they had been associated with the first magnetic resonance data during the spectral decomposition. Sharp edges owing to anatomical structures are consequently retained, with noise at the same time being reduced, in particular in regions of low signal intensity. This aspect accordingly makes a separated intensity correction of noise possible and prevents an amplification of the noise in portions with low signal intensity and/or portions with high first correction values in the first homogenization field. This aspect thus makes particularly good homogenization of the magnetic resonance data possible.

One aspect of the method provides that the mapping rule is linear at least in sections. According to this aspect, the derivation and/or the gradient of the mapping rule is constant in individual sections. A mapping rule of this kind can be implemented particularly intuitively and precisely by means of threshold values separating the individual sections. In addition, the slope of the mapping rule can be determined in accordance with the second signal intensities of the second magnetic resonance data. A mapping rule of this kind can be effectively determined on the basis of a few parameters.

Furthermore, the aspects of the disclosure start from an image processing unit with an input, a differentiation unit, a determination unit, a homogenization unit, and an output. The image processing unit is embodied to carry out an inventive method for generating homogenized magnetic resonance data.

For this, the image processing unit typically has a processor unit. The image processing unit can be provided with the magnetic resonance data of an examination object, the first homogenization field specific to the examination object, the frequency spectrum, and/or at least two frequency ranges via the input. The image processing unit can be provided with further functions, algorithms, or parameters required in the method via the input. The homogenized magnetic resonance data and/or the second homogenization field and/or the at least two frequency ranges and/or the mapping rule and/or further results of an aspect of the inventive method can be provided via the output.

The differentiation unit is embodied for a spectral decomposition of the magnetic resonance data into the at least two frequency ranges. The determination unit is embodied for determining the second homogenization field by taking into account the first homogenization field.

The homogenization unit is embodied for
applying the first homogenization field to the first magnetic resonance data and generating first homogenized magnetic resonance data,
applying the second homogenization field to the second magnetic resonance data and generating second homogenized magnetic resonance data,
generating homogenized magnetic resonance data by combining the first homogenized magnetic resonance data with the second homogenized magnetic resonance data.

The image processing unit can also comprise a function-determining unit, which function-determining unit is embodied for determining and/or providing the mapping rule.

The image processing unit can be integrated in a magnetic resonance device, which magnetic resonance device is embodied for acquisition of the magnetic resonance data of the examination region. The image processing unit can be connected to such a magnetic resonance device. The image processing unit can also be installed separately from the magnetic resonance device.

Aspects of the inventive image processing unit are embodied analogously to the aspects of the inventive method. The image processing unit can have further control components that are necessary and/or advantageous for carrying out an inventive method. The image processing unit can also be embodied to send control signals and/or to receive and/or process control signals in order to carry out an inventive method. Computer programs and further software can be saved on a memory unit of the image processing unit, by means of which the processor unit of the image processing unit automatically controls and/or carries out a process sequence of an inventive method.

An inventive computer program product can be loaded directly in a memory unit of a programmable image processing unit and has program code means in order to carry out an inventive method when the computer program product is executed in the image processing unit. The inventive method can consequently be carried out quickly, in an identically repeatable manner and robustly. The computer program product is configured such that it can carry out the inventive method steps by means of the image processing unit. The image processing unit must in each case have the prerequisites such as, for example, an appropriate main memory, an appropriate graphics card or an appropriate logic unit, so the respective method steps can be carried out efficiently. The computer program product is saved, for example, on an electronically readable medium or stored on a network or server from where it can be loaded into the processor of a local computing unit which is directly connected to the image processing unit or can be embodied to be part of the image processing unit. Furthermore, items of control information of the computer program product can be saved on an electronically readable data carrier. The items of control information of the electronically readable data carrier can be configured in such a way that they carry out an inventive method when the data carrier is used in a computing unit of an image processing unit. Examples of electronically readable data carriers are a DVD, a magnetic tape or a USB stick, on which electronically readable items of control information, in particular software, is saved. When these items of control information (software) are read from the data carrier and saved in a control unit and/or image processing unit, all inventive aspects of the methods described above can be carried out.

Furthermore, the aspects of the disclosure start from an electronically readable data carrier on which a program is stored which is provided for carrying out a method for generating homogenized magnetic resonance data.

The advantages of the inventive image processing unit, the inventive computer program product, and the inventive electronically readable data carrier substantially correspond to the advantages of the inventive method for generating homogenized magnetic resonance data, which are stated above in detail. Features, advantages, or alternative aspects mentioned in this connection can likewise also be transferred to the other claimed subject matters, and vice versa.

### BRIEF DESCRIPTION OF THE DRAWINGS

Further advantages, features, and details of the disclosure can be found in the exemplary aspects described below, as well as on the basis of the drawings.

In the drawings:

FIG. 1 shows a flowchart of a first aspect of an inventive method,

FIG. 2 shows a flowchart of a second aspect of an inventive method,

FIG. 3 shows a first aspect of a mapping rule,

FIG. 4 shows a second aspect of a mapping rule,

FIG. 5 shows a third aspect of a mapping rule,

FIG. 6 shows a third aspect of a mapping rule, and

FIG. 7 shows an inventive image processing unit in a schematic representation.

### DETAILED DESCRIPTION

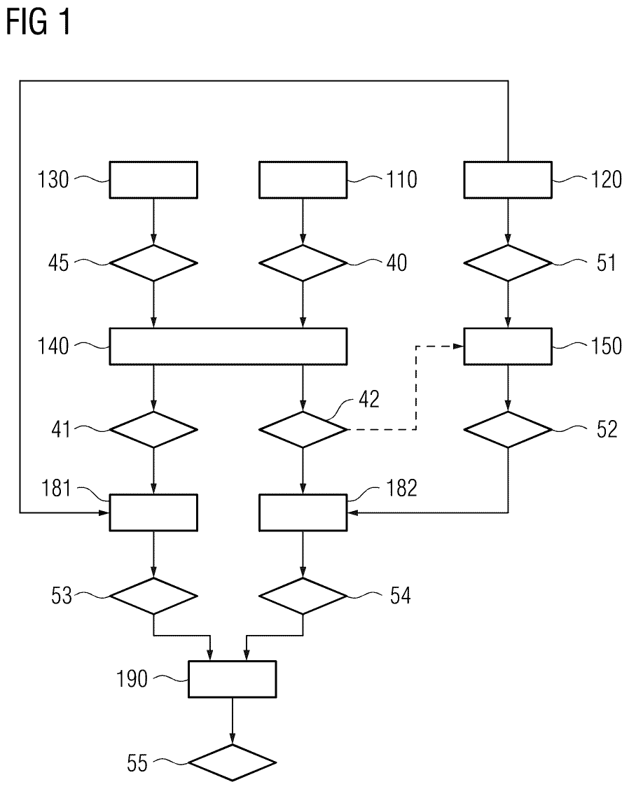

FIG. 1 shows a flowchart of a first aspect of an inventive method for generating homogenized magnetic resonance data 55. Method step 110 provides for the provision of magnetic resonance data 40 of an examination object.

In method step 120, a first homogenization field 51 specific to the examination object is provided. Method step 130 provides for the provision of a frequency spectrum 45 having at least two frequency ranges 46, 47. In method step 140, the magnetic resonance data 40 is spectrally decomposed into the at least two frequency ranges 46, 47, with first magnetic resonance data 41 being associated with a first frequency range 46 of the at least two frequency ranges 46, 47 and second magnetic resonance data 42 being associated with a second frequency range 47 of the at least two frequency ranges 46, 47.

Method step 150 comprises determining a second homogenization field 52 by taking into account the first homogenization field 51. This can optionally occur by taking into account the second magnetic resonance data 42.

In method step 181, the first homogenization field 51 is applied to the first magnetic resonance data 41, whereby first homogenized magnetic resonance data 53 is generated.

Method step 182 provides for applying the second homogenization field 52 to the second magnetic resonance data 42, whereby second homogenized magnetic resonance data 54 is generated. Method step 190 comprises generating homogenized magnetic resonance data 55 by combining the first homogenized magnetic resonance data 53 with the second homogenized magnetic resonance data 54.

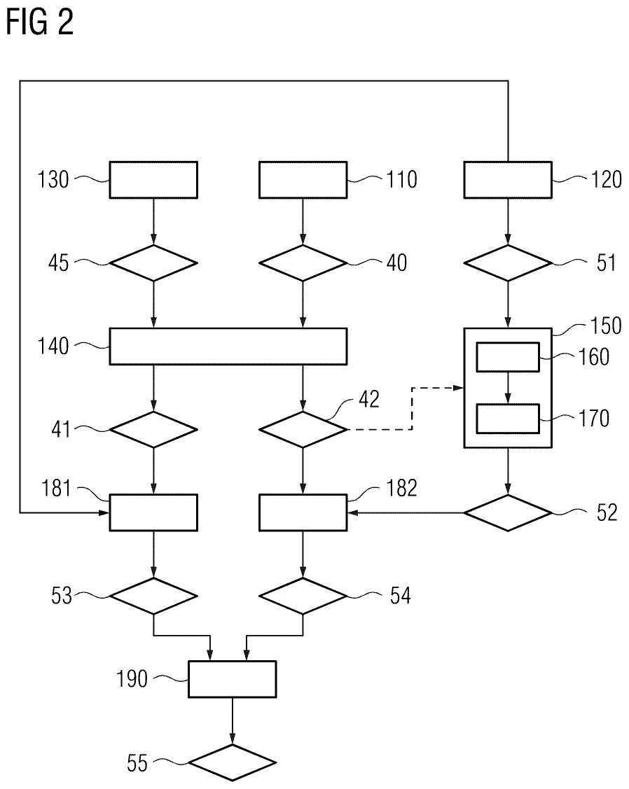

FIG. 2 shows a flowchart of a second aspect of an inventive method. The second aspect differs from the first aspect represented in FIG. 1 in that determining the second homogenization field in method step 150 comprises further method steps 160, 170. Method step 160 thus provides for the provision of a mapping rule 60, which mapping rule 60 maps first correction values of the first homogenization field 51 to second correction values. Method step 170 comprises generating the second homogenization field 52 by applying the mapping rule 60 to the first homogenization field 51. In method step 160, the second magnetic resonance data 42 can be taken into account.

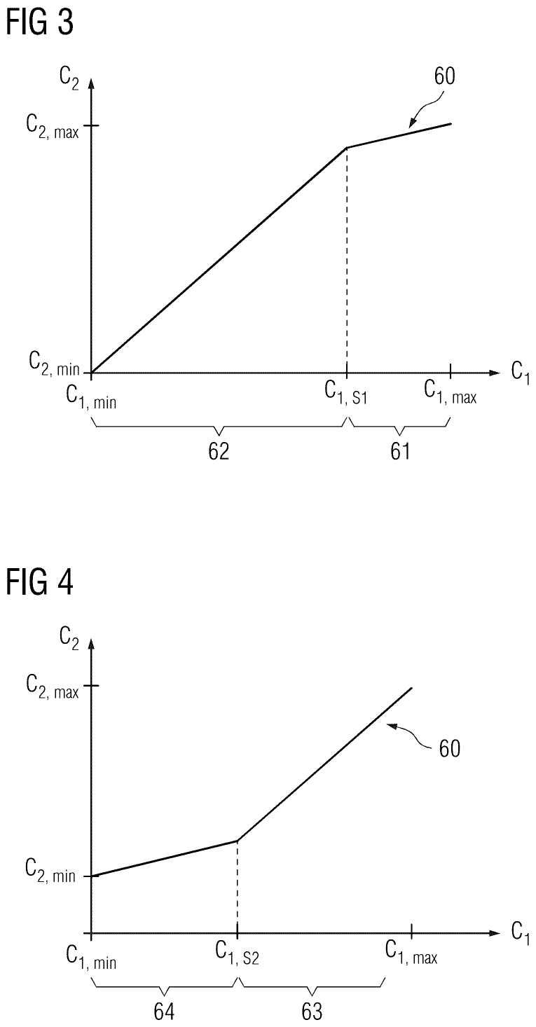

FIG. 3 shows a first aspect of a mapping rule 60 which maps the first correction values C₁ of the first homogenization field 51 to second correction values C₂. The first value range [C_{1,min}; C_{1,max}] comprises all first correction values C1 as are provided for the first homogenization field 51. These are represented in FIG. 3 as the abscissa. The first value range [C_{1,min}; C_{1,max}] is broken down into a first section 61 and into a second section 62. The first section 61 comprises part of the upper third of the first value range [C_{1,min}; C_{1,max}]. The second section 62 comprises at least one middle region of the first value range [C_{1,min}; C_{1,max}], in the represented case the first value range [C_{1,min}; C_{1,max}] minus the first section 61. The first section 61 and the second section 62 are separated from each other by the first threshold value C_{1,S1}. The mapping rule 60 describing the dependence of the second correction value C2, plotted on the ordinate, has a lower slope in the first section 61 than in the second section 62. The second value range [C_{2,min}; C_{2,max}] comprising the second correction values C₂ is smaller than the first value range [C_{1,min}; C_{1,max}] hereby. The mapping rule 60 is, according to the one in this representation, embodied to be linear in sections. A continuous, in particular fluent, transition between the first section 61 and the second section 62 is likewise conceivable, so the second derivation at the first threshold value C_{1,S1} can be different to zero.

FIG. 4 shows a second aspect of a mapping rule that maps the first correction values C1 of the first homogenization field 51 to the second correction values C₂. The first value range [C_{1,min}; C_{1,max}] is broken down into a third section 63 and into a fourth section 64. The fourth section 64 comprises part of the lower third of the first value range [C_{1,min}; C_{1,max}]. The third section 63 comprises at least one middle region of the first value range [C_{1,min}; C_{1,max}], in the represented case the first value range [C_{1,min}; C_{1,max}] minus the fourth section 64. The third section 63 and the fourth section 64 are separated from each other by the second threshold value C_{1,S2}. The mapping rule 60 describing the dependence of the second correction value C₂, plotted on the ordinate, associates second correction values C₂ with the first correction values C₁ of the fourth section 64, which are greater than the underlying first correction values C₁. This is implemented in the represented aspect by a mapping rule 60 which in the fourth section 64 has a lower slope than in the third section 63 and at the same time is embodied to be steady at the transition from the third section 63 to the fourth section 64. The second value range [C_{2,min}; C_{2,max}] comprising the second correction values C₂ is smaller than the first value range [C_{1,min}; C_{1,max}] hereby. The mapping rule 60 is, according to the one in this representation, embodied to be linear in sections. A continuous, in particular fluent, transition between the third section 63 and the fourth section 64 is likewise conceivable, so the second derivation at the second threshold value C_{1,S2} can be different to zero.

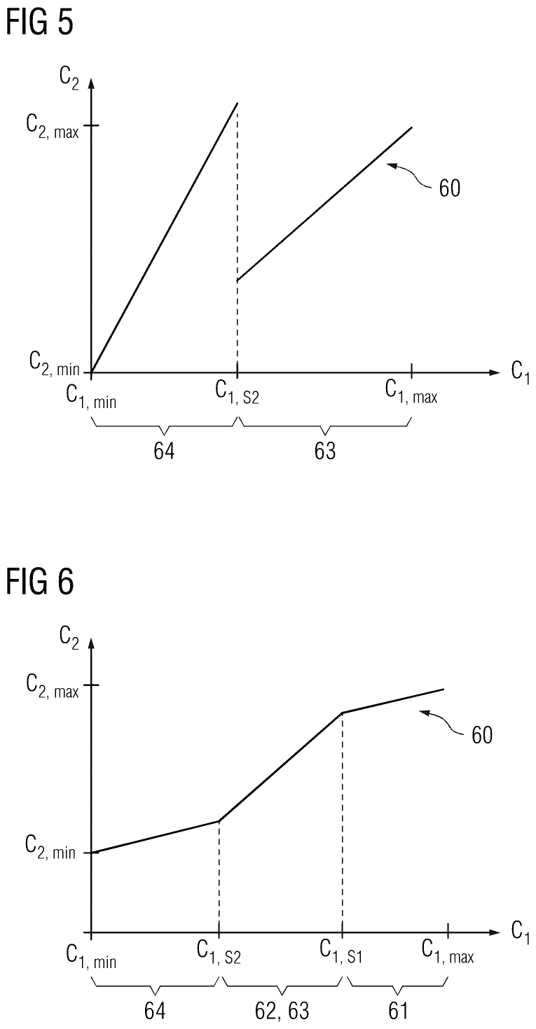

FIG. 5 shows a third aspect of a mapping rule which maps the first correction values C₁ of the first homogenization field 51 to second correction values C₂. The mapping rule 60 according to this third aspect associates second correction values C₂ with first correction values C₁ of the fourth section 64, which are greater than the underlying first correction values C₁. Contrary to the second aspect represented in FIG. 4, this mapping rule 60 at the transition from the third section 63 to the fourth section 64 is discontinuous, and the gradient of the mapping rule 60 is greater in the fourth section than the gradient of the mapping rule 60 in the third section. In particular, the slope of the mapping rule 60 according to this third aspect is two in the fourth section 64 and one in the third section 63.

FIG. 6 shows a fourth aspect of a mapping rule 60 which combines the first aspect of FIG. 3 and the second aspect of FIG. 4. In this case, the third section 63 corresponds to the second section 62, so the mapping rule 60 comprises at least three sections 61, 62, 63, 64, with the slope of the mapping rule 60 in the first section 61 and in the fourth section 64 being lower than in the middle section 62, 63. The mapping rule 60 is, according to the one in this representation, embodied to be linear in sections. A continuous, in particular fluent, transition between the individual sections 61, 62, 63, 64 is likewise conceivable, so the second derivation at the first threshold value C_{1,S1} and/or at the second threshold value C_{1,S2} can be different to zero.

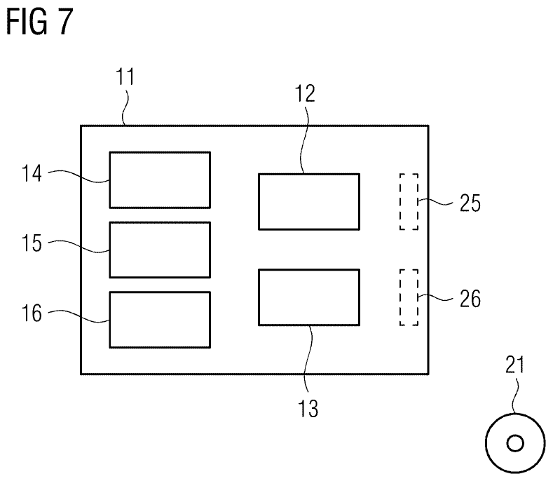

FIG. 7 shows an image processing unit 11 for carrying out an inventive method in a schematic representation. For this, the image processing unit 11 has an input 12, a differentiation unit 14, a determination unit 15, a homogenization unit 16, and an output 13. The image processing unit 11 can comprise a function-determining unit and/or memory unit and/or computing unit (not represented). The image processing unit 11 optionally has a display unit 25. The homogenized magnetic resonance data 55 and/or the at least two frequency ranges 46, 47 and/or the mapping rule 60 can be displayed on the display unit 25, for example, on at least one monitor, for a user. In addition, the image processing unit 11 optionally has an input unit 26 by means of which items of information can be input by a user and/or the at least two frequency ranges 46, 47 of the frequency spectrum 45 can be specified.

The differentiation unit 14 is embodied for carrying out method step 140, the spectral decomposition of the magnetic resonance data 40 into the at least two frequency ranges 46, 47. The determination unit 15 is embodied for carrying out method step 150, determining the second homogenization field 52 by taking into account of the first homogenization field 51. The homogenization unit 16 is embodied for carrying out method steps 181, 182, 190. The homogenized magnetic resonance data 55 can be provided via the output 13. Method steps 110, 120, 130 can be carried out via the input 12.

In addition, the image processing unit 11 has computer programs and/or software which can be loaded directly into a memory unit (not represented) of the image processing unit 11, with program means in order to carry out a method for generating homogenized magnetic resonance data 55 when the computer programs and/or software are executed in the image processing unit 11. For this, the image processing unit 11 has a processor (not represented) which is configured to execute the computer programs and/or software. Alternatively, the computer programs and/or software can also be saved on an electronically readable data carrier 21, embodied separately from the image processing unit 11, with it being possible for the image processing unit 11 to access data on the electronically readable data carrier 21 via a data network.

The represented image processing unit 11 can, of course, comprise further components that image processing units 11 usually have. A general mode of operation of an image processing unit 11 is known to a person skilled in the art, moreover, so a detailed description of the further components will be omitted.

A method for generating homogenized magnetic resonance data 55 can also be present in the form of a computer program product, which implements the method on the image processing unit 11 when it is executed on the image processing unit 11. An electronically readable data carrier 21 with electronically readable items of control information stored thereon can likewise be present, which items comprise at least one such computer program product just described and are embodied in such a way that they carry out the described method when the data carrier 21 is used in an image processing unit 11.

Although the aspects of the disclosure have been illustrated and described in detail by the preferred exemplary aspects, it is not restricted by the disclosed examples and a person skilled in the art can derive other variations herefrom without departing from the scope of the invention. Independent of the grammatical term usage, individuals with male, female or other gender identities are included within the term.

1. A method for generating homogenized magnetic resonance data, the method comprising:
providing magnetic resonance data of an examination object;
providing a first homogenization field specific to the examination object;
providing a frequency spectrum having at least two frequency ranges;
spectral decomposition of the magnetic resonance data into the at least two frequency ranges, wherein first magnetic resonance data is associated with a first frequency range of the at least two frequency ranges and second magnetic resonance data is associated with a second frequency range of the at least two frequency ranges;
determining a second homogenization field by taking into account the first homogenization field;
applying the first homogenization field to the first magnetic resonance data and generating first homogenized magnetic resonance data;
applying the second homogenization field to the second magnetic resonance data and generating second homogenized magnetic resonance data; and
generating homogenized magnetic resonance data by combining the first homogenized magnetic resonance data with the second homogenized magnetic resonance data.

2. The method as claimed in claim 1, wherein the first frequency range has lower frequencies than the second frequency range.

3. The method as claimed in claim 1, wherein the second homogenization field is determined by taking into account the second magnetic resonance data.

4. The method as claimed in claim 1, wherein determining the second homogenization field comprises:
providing a mapping rule mapping first correction values of the first homogenization field to second correction values; and
generating the second homogenization field by applying the mapping rule to the first homogenization field.

5. The method as claimed in claim 4, wherein the mapping rule is provided by taking into account the second magnetic resonance data.

6. The method as claimed in claim 4, wherein a first value range comprising the first correction values comprises a first section and a second section, the first section comprises greater first correction values than the second section and a gradient of the mapping rule in the first section is smaller than a gradient of the mapping rule in the second section.

7. The method as claimed in claim 6, wherein the first section comprises at least one portion of an upper third of the first value range and/or the second section comprises at least one portion of the middle third of the first value range.

8. The method as claimed in claim 4, wherein a first value range comprising the first correction values comprises a third section and a fourth section, the third section comprises greater first correction values than the fourth section and the mapping rule is embodied in such a way that the second correction values of the first section associated with the first correction values are greater than the corresponding first correction values of the first section.

9. The method as claimed in claim 8, wherein a gradient of the mapping rule in the fourth section is greater than a gradient of the mapping rule in the third section.

10. The method as claimed in claim 8, wherein the fourth section comprises at least one portion of the lower third of the first value range and/or the third section comprises at least one portion of the middle third of the first value range.

11. The method as claimed in claim 6,
wherein the first value range comprising the first correction values comprises a third section and a fourth section, the third section comprises greater first correction values than the fourth section and the mapping rule is embodied in such a way that the second correction values of the first section associated with the first correction values are greater than the corresponding first correction values of the first section, and
wherein the third section corresponds to the second section.

12. The method as claimed in claim 4, wherein the mapping rule is linear at least in sections.

13. An image processing unit, comprising:
an input;
a differentiation unit;
a determination unit;
a homogenization unit; and
an output,
which is configured to carry out a method for generating homogenized magnetic resonance data as claimed in claim 1.

14. A non-transitory electronically readable data carrier on which a program is stored which is embodied in such a way that the program carries out the method for generating homogenized magnetic resonance data as claimed in claim 1 when the data carrier is used in an image processing unit.
